(19) United States
(12) Patent Application Publication
(10) Pub. No.: US 2004/0157237 A1
(43) Pub. Date: Aug. 12, 2004

(54) Optochemical sensing with multi-band fluorescence enhanced by surface plasmon resonance

(75) Inventors: Henryk Malak, Ellicott City, MD (US); Valery Bogdanov, Lawrenceville, NJ (US)

Correspondence Address: Henryk Malak, 8444 High Ridge Road, Ellicott City, MD, 21043, US

(73) Assignee: Americal Environmental Systems, Inc.

(21) Appl. No.: 10/656,629

(22) Filed: Sep. 8, 2003

Related U.S. Application Data
(60) Provisional application No. 60/446,096, filed on Feb. 10, 2003.

(51) Int. Cl. C12Q 1/68; G01N 33/53

(52) U.S. Cl. 435/6; 435/7.1

(57) ABSTRACT

This invention relates to methods and compositions of an optochemical absorption and fluorescence sensing of materials for molecular identification and measuring the concentration of one or more analytes in the sample. The methods and compositions of an enhanced absorption and fluorescence multibands of a molecule by surface plasmon resonance of metal nanoparticles are described. The invention expands the analytical capacity of conventional, single-band absorption and fluorescence spectroscopy and sensing through implementation of the method of enhanced multi-band absorption and fluorescence of higher excited states (HES) and lowest excited state (LES) of the same molecule when the molecule is in close proximity to metal nanoparticles. The method provides a band-selective enhancement of a low quantum yield emission of HES fluorescence that leads to easy-to-detect multi-band fluorescence.

### CROSS REFERENCE TO RELATED APPLICATIONS

This application is related to U.S. Provisional Patent Application No. 60/446,096, entitled “Optochemical Sensing with Multi-Band Fluorescence Enhanced by Surface Plasmon Resonance” filed Feb. 10, 2003, which is herein incorporated by reference.

### STATEMENT REGARDING FEDERALLY SPONSORED RESEARCH

There is NO claim for federal support in research or development of this product.

### REFERENCES CITED

The following are patents found that may be associated with this information.

U.S. Patent Documents

| | | |
|---|---|---|
| U.S. Pat. No. 5,866,433 | Feb. 2, 1999 | Schalkhammer, et al. |
| U.S. Pat. No. RE37,412 | Oct. 16, 2001 | Schalkhammer, et al. |

### BACKGROUND OF THE INVENTION

This invention relates to an optochemical sensing of materials for molecular identification and measuring the concentration of one or more analytes in the sample.

Optochemical sensing is based on reading an optical signal generated by a sensor interacting with an analyte. Fluorescence sensors as a group are the most sensitive optochemical sensors that utilize a fluorescence signature of sensor and/or analyte (excitation and/or emission spectra, intensity, lifetime, polarization) to identify materials with high specificity. The invention discovers new principles of fluorescence sensing that improves fluorescence sensor specificity, sensitivity and response time, dramatically. The invention will be applied to life sciences, biomedicine, defense, and material science research and application.

### DESCRIPTION OF THE PRIOR ART

Schalkhammer, et al., U.S. Pat. No. 5,866,433. An optochemical fluorescence sensor with a biorecognitive layer for measuring the concentration of one or more analytes in a sample is provided with at least one island layer that is applied on a sensor substrate. The islands of the island layer are in the form of electrically-conductive material and have a diameter of less than 300 nm, the biorecognitive layer being directly applied on the island layer or bound via a spacer film. In addition, an analyte-specific fluorescent compound is provided which may be added to the sample or is provided in the sensor itself. The biorecognitive layer can bind the analyte to be measured directly or by means of analyte-binding molecules, the originally low quantum yield of the fluorescent compound increasing strongly in the vicinity of the island layer.

Schalkhammer, et al., US RE37,412. An optochemical sensor for measuring concentrations of analytes is provided with a reactive matrix preferably made of polymeric material capable of swelling. Further provided are a mirror layer and a layer of a plurality of discrete islands that are electrically conductive, between which layers the reactive matrix is positioned, the diameter of the islands being smaller than the wavelength of the light employed for monitoring and evaluation.

### SUMMARY OF THE INVENTION

The objective of the invention is to provide a method for the optochemical fluorescence sensing of fluorophores and/or analytes, which will allow very sensitive, up to single molecule specific, fast identification of the fluorophore and/or will aid in measure of analyte concentration.

In the invention this objective is achieved by measurement and analysis of multiband emission of the fluorophore enhanced by electromagnetic fields of surface plasmon resonance (SPR) from nearby metal nanoparticles.

Multiband fluorescence includes emission bands from low excited state (LES) and higher excited states (HES) of the same analyte molecule (FIG. 1). As a rule, HES emission is difficult to measure due to ultra-fast HES nonradiative decay and very low quantum yield of HES fluorescence (<1%, typically). All current fluorescence sensors employ measurement at a single-band LES fluorescence of analyte.

The invention expands an analytical capacity of conventional, single-band fluorescence spectroscopy and sensing through implementation of the method to measure enhanced multi-band—HES and LES fluorescence bands of the same fluorophore. The method provides a band-selective enhancement of a low quantum yield emission of HES fluorescence that leads to easy-to-detect multi-band fluorescence sensing.

The invention employs a dependence effect of fluorophore emission rate enhancement by nearby metal (silver, gold) nanoparticles on quantum yield of fluorophore (Q). If the fluorophore is positioned inside SPR evanescent wave zone, strong electromagnetic fields generated by surface plasmons of nanoparticles, enhance fluorophore absorption and emission rate 100 s-1000 s folds, respectively. This leads to an enhanced fluorescence quantum yield and measured intensity. However, enhanced quantum yield is limited by a maximum Q value equal to 100%. This limitation results in a relatively low efficiency of intensity enhancement for a fluorophore with high Q value; but, it does not place a practical limit on the enhanced emission intensity of a fluorophore with low Q value.

Experimental data confirms this conclusion: the emission intensity measured for a series of fluorophores in the vicinity of metal nanoparticles was greatly increasing for the decreasing values of Q for fluorescence (FIG. 2, graph based on data published by J. R. Lakowicz et al. “Radiative Decay Engineering. 2. Effects of Silver Island Films on Fluorescence Intensity, Lifetimes, and Resonance Energy Transfer”, Anal. Biochem., 301:261 (2002)). Therefore, SPR-mediated growth of emission intensity is expected to be high for excited electronic state with low quantum yield (HES) and low for state with a high quantum yield (LES) of the same fluorophore. As a result, the fluorescence intensities from the HES and LES would reach comparable levels. Thus, at enhanced fluorescence with metal nanoparticle, HES fluorescence could be used as an additional, measurable optical signature of fluorophore.

The effect of fluorescence enhancement depends on a distance between fluorophore and nanoparticle. As the distance increases, enhance fluorescence signal decreases, and a significant enhancement is available for a fluorophore positioned inside an evanescent wave zone of surface plasmon, only. On the other side, direct metal-fluorophore contact energy transfer quenches fluorescence completely. Ultra-thin dielectric barrier-spacer (plastic, polymer or SiO₂ layer with thickness above 10 nm) between nanoparticle and fluorophore eliminates quenching and causes significant fluorescence enhancement. The upper limit of the layer thickness for fluorescence enhancement depends on the depth of evanescent light penetration in the optochemical sensor. According a recent paper of Horváth and et al., a refractive index of planar waveguide can increase the penetration of evanescent zone up to a few microns (Horváth et al. “Optical waveguide sensor for on-line monitoring of bacteria”, Optics Letter, 28, 1233 (2003)).

Additionally, molecular probes designed to capture analyte in addressable location (microarray) could be developed on spacer surface. It leads to a sensor design capable of simultaneous and highly parallel multi-band fluorescence sensing of analytes.

Thus, a sensor in the invention comprises of:

a) a support with designed refractive index of used material

b) a thin film of metal nanoparticles or metal islands placed on a surface of support

c) a dielectric or biorecognitive barrier with thickness more than 10 nm placed on a nanoparticle coated substrate

d) analyte probes attached or not attached to an external barrier surface (microarray or other format)

e) an excitation source: optical illumination or chemical reaction

The invention can also be applied to sensor design based on single metal nanoparticle or an assembly of metal nanoparticles, where each nanoparticle is coated or not coated with dielectric or biorecognitive barrier, dispersed in medium, cells, or other sensing materials and excited with light causing surface plasmon enhanced emission of surrounding fluorophores.

Hyperspectral imaging is a preferable method to measure and analyze fluorescence of analytes immobilized on a sensor surface. FIG. 3 shows a possible schematic of proposed sensor and FIG. 4 shows a hyperspectral imager (HIS) employing the effect of slit-free, optical-rotation dispersion on polychromatic radiation (P. Herman et al. “Compact hyperspectral imager for low light applications” SPIE Proc. 2001, 4259, pp. 8-16). However, other optical techniques can be also applied with optochemical multiband enhanced fluorescence sensing, like time-resolved spectroscopy, fluorescence polarization, fluorescence recovering after photobleaching, fluorescence resonance energy transfer surface, enhanced multiband Raman scattering (but not limited to them). The multi-band enhanced emission can be generated by electromagnetic radiation source in single, and multi-photon and/or nonlinear optical modes of excitation. It can be also generated by chemiluminescence, electro-optically, electrochemically and other luminescence techniques. In all of these methods, band-selective intensity enhancement leads to comparable intensity HES and LES bands.

Thus, hyperspectral detection and other above mentioned techniques could be used for optochemical sensing employed multi-band enhanced emission.

### BRIEF DESCRIPTION OF THE DRAWINGS

FIG. 1. Schematic diagram of the fluorophore electronic states, processes (left) and fluorescence spectra (right). 1 and 2—one photon absorption/LES and HES population, 1+3—two-photon step wise absorption/HES population, 4—LES fluorescence, 5—HES fluorescence, 6—LES nonradiative decay, 7—HES nonradiative decay.

FIG. 2. Dependence of enhanced fluorescence intensity with nearby silver nanoparticle on fluorophore quantum yield.

FIG. 3. Schematic diagram of the proposed sensor and hyperspectral optical setup.

SENSOR (insert). Metal nanoparticles (tens of nanometers in diameter) are placed on the surface of glass substrate (prism). Nanoparticle layer is coated with a 10-100 s nm thick dielectric layer (polymer or SiO₂) to create a physical barrier between the metal particles and a fluorophore. Microarray of analyte captured spots is attached to a surface of dielectric layer. The excitation can be delivered via evanescent wave coupling using the effect of total internal reflection at the prism surface.

OPTICAL SET-UP. The entire microarray can be illuminated with laser pulses at two different wavelengths. To produce both LES and HES signatures, the sample is simultaneously illuminated by two nanosecond laser pulses at different wavelengths, for example 4^{th} harmonics (266 nm) and fundamental (1064 μm) wavelength of Nd:YAG laser. The conventional (LES) fluorescence spectrum will be acquired following single-photon excitation at 266 nm. To obtain HES fluorescence spectrum, two-photon resonant (step-wise) excitation is used. In the first step, LES is populated when the molecules in their ground electronic state absorb a photon at 266 nm In the second step the excited molecules in LES absorb the second photon at 1064 nm; this results in population of HES. The measured HES fluorescence spectrum is blue-shifted compared to the LES fluorescence. To obtain the full analyte optical signature (LES+HES fluorescence), many other combinations can be used in the step-wise excitation. A Nd:YAG laser equipped with a standard set of nonlinear crystals can generate pulses at the fundamental frequency plus four harmonics.

In this example, the output a Q-switched Nd:YAG laser (5 ns pulses, up to 100 Hz repetition rate), consists of the fundamental (1064 nm) and 2^{nd} harmonics (532 nm) and/or 3^{rd} harmonics (355 nm, and/or 4^{th} harmonics (266 nm). This multitude of wavelength provides a high degree of flexibility in detection of practically any organic/inorganic matter. The fundamental output is divided into two beams by means of an 60/40 beam splitter. The 40% fraction of the 1064 nm beam passes through a an assembly of nonlinear crystals and is converted into the harmonics which are directed into the total internal reflection (TIR) prism made of fused silica. The harmonics illuminate the glass-sensor interface at the critical angle and excite the bio-agent fluorophores attached (captured) to the microarray via evanescent wave illumination. The remaining 60% of the fundamental (1064 nm) enters the TIR prism from the opposite prism side and overlaps with the harmonics beam at the glass-sensor interface. A shutter placed in the fundamental beam controls the excitation scheme by blocking passing the 1064 nm radiation Microarray emission is collected by infinity-corrected lens and transmitted through laser cut-off filter and the HSI module. An imaging lens produces a microarray spectral image which is then captured by a cooled CCD array.

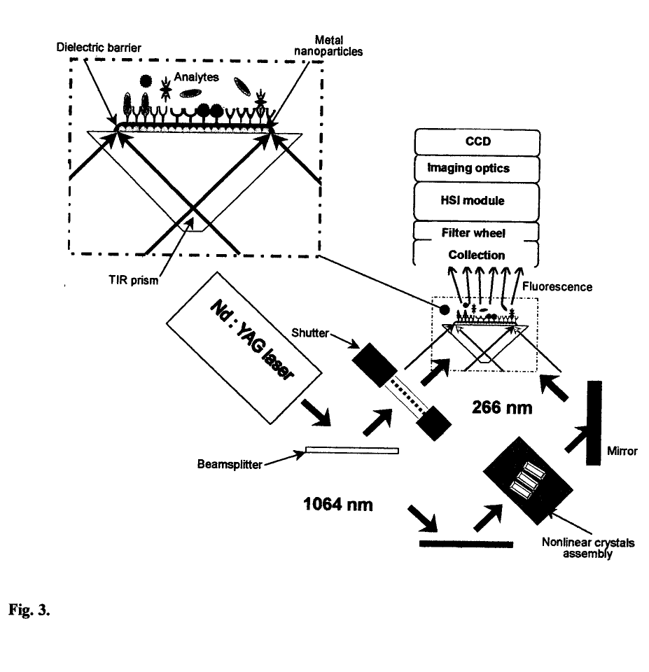

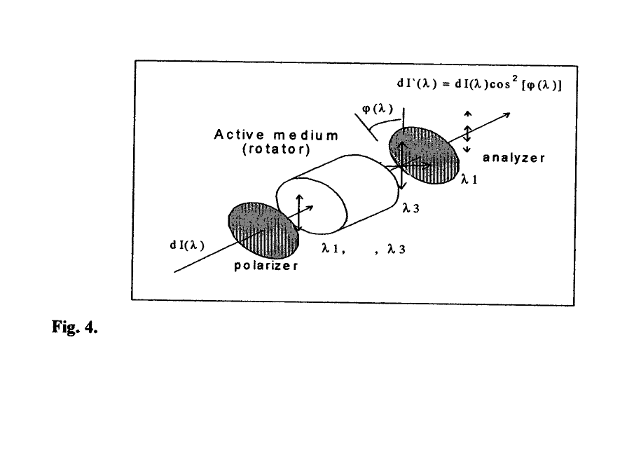

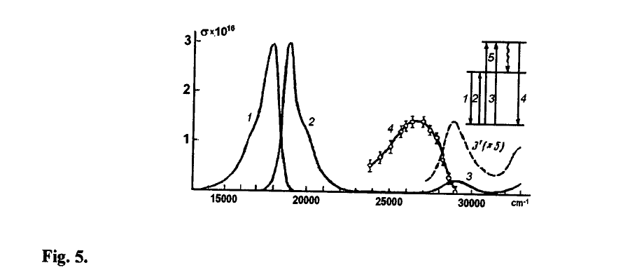

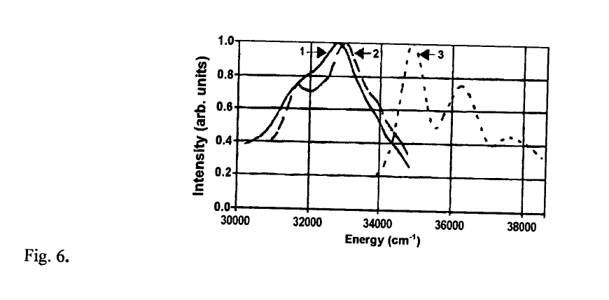

FIG. 4. Schematic diagram of hyperspectral imager (HIS) module in FIG. 3.

This module utilizes the effect of optical rotation dispersion (ORD) on polychromatic light. It consists of a polarization rotator—an optically active medium (crystalline quartz) placed between a pair of polarizers with their transmission axes aligned parallel to each other. The polarization direction of linearly polarized input light rotates during propagation through the rotator and the rotation angle depends on the wavelength and the rotation power of the optical rotator. Due to the ORD effect, the polarization planes of different spectral components become angularly dispersed after passage through the rotator. The emerging light is partially blocked by the output polarizer and the attenuation of the light at different wavelengths is determined by the material-dependent ORD function. Each wavelength component contributes to every point in the image according to the cosine square of the angle between the polarization of the rotated wavelength component and the fixed output polarization analyzer.

FIG. 5. Absorption and fluorescence spectra of Rhodamine 6G solution: (1) LES fluorescence, (2) long-wavelength absorption spectrum, (3) short-wavelength absorption spectrum, (4) HES fluorescence. (5) absorption of excited molecules. Upper right: scheme of molecular states and optical processes.

FIG. 6. Absorption spectrum (3); and HES fluorescence spectra of benzanthracene solution at stepwise (ω₁+ω₂) excitation with (1) ω₁=18,800 cm⁻¹ and (2) ω₂=14,400 cm⁻¹.

### DETAILED DESCRIPTION OF THE INVENTION

Current fluorescence techniques, despite their relatively high sensitivity, are restricted by fundamental photo-physical processes. For certain fluorophores, fluorescence might not be sufficiently sensitive to be used for successful identification of single-particle samples. For example, the typical fluorescence spectra of bacteria do not always provide sufficiently selective signature of pathogens (R. G. Pinnick, et al., “Real-time measurement of fluorescence spectra from single airborne biological particles”, Field Anat. Chem. Technol. 3, 221 (1999); Scully et al., “FAST CARS: Engineering a laser spectroscopic technique for a rapid identification of bacterial spores”. PNAS, 99, 10994, (2002)).

The invention provides a novel methodology that overcomes limitations of the conventional fluorescence sensoring. To increase the fluorescence intensity, we will employ the effect of enhanced fluorophore absorption/emission rates by surface plasmon resonance (SPR) of nearby metal (silver, gold) nanoparticles (M. Kerker, “Optics of colloid silver”, J. Colloid Interface Sci. 105, 298 (1985); Lakowicz et al, “Intrinsic fluorescence from DNA can be enhanced by metallic particles”, Biochem. Biophys. Res. Comm. 286, 875 (2001); Gryczynski et al., “Multiphoton excitation of fluorescence near metallic particles: enhanced and localized excitation”, J. Phys. Chem. B, 106, 2191 (2002)). When the fluorophore is in a direct contact with a metal nanoparticle, fluorescence is completely quenched by energy transfer to metal. However, at the distance of 10 nm-100 s nm between the fluorophore and metal nanoparticle the absorption and emission rates can be, respectively, enhanced by factors of ˜10² and ˜10³ [11]. The enhancement of the emission intensity depends on fluorescence quantum yield Q, where 0≦Q≦1.

It is the first invention that implements a measurement of multi-band fluorescence for analyte identification in fluorescence sensing. Current fluorescence sensors are based on a fundamental principle of molecular fluorescence known as Kasha rule (M. Kasha, “Characterization of electronic transitions in complex molecules”, Discuss. Faraday Soc., 8, 14 (1950)). According to the Kasha rule, a fluorophore in the condensed phase emits a single-band spectrum from its lowest singlet excited state (LES), due to the vibrational relaxation and non-radiative dissipation of excitation energy. Natural emission rate for a fluorophore (<10⁹ s⁻¹) defined by fluorophore transient dipole puts a limit on a rate for fluorophore nonradiative decay of measured fluorescence.

Fluorescence from high-excited state (HES) can provide additional to LES fluorescence information about molecular structure of analyte in question. However, the non-radiative decay of the high-excited state is thousands of times faster than HES radiative decay, which leads to a very low Q for the HES emission (much lower than for the LES emission) and difficulties in detection of HES fluorescence. The ratio of Q for LES to HES fluorescence may be as high as 10⁵ (Bogdanov, “Fluorescence and multiwave mixing induced by photon absorption of excited molecules”, Topics in Fluorescence Spectroscopy, Vol. 5: Nonlinear and Two-photon induced Fluorescence, Ed. J. Lakowicz, Plenum Press, 1997; Galanin et al. “Fluorescence from the second excited electronic level and absorption by excited R6G molecules”, Bull. Acad. Sc., Phys. Ser. 36, 850 (1972)).

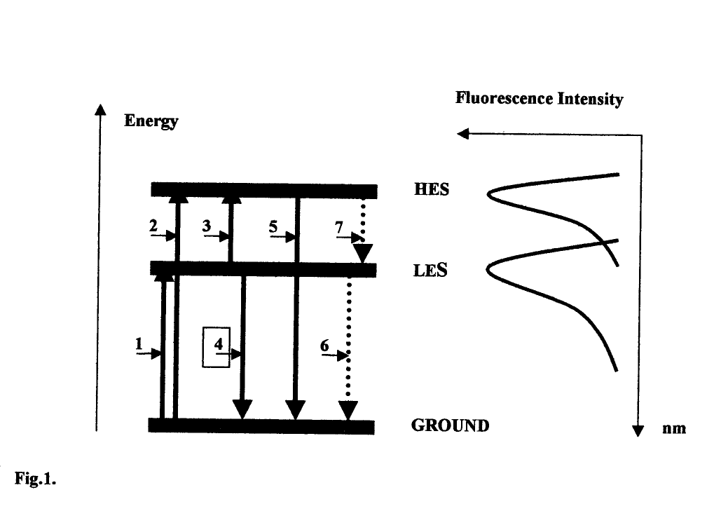

Although HES fluorescence is not available in current fluorescence sensing, its characteristics have sensitivity to both the excitation energy and the fluorophore's chemical environment. The normally low value of Q prevents the multi-band HES fluorescence from being used as a very selective optical signature. FIG. 1 shows fluorophore electronic states and origin of LES and HES fluorescence spectra.

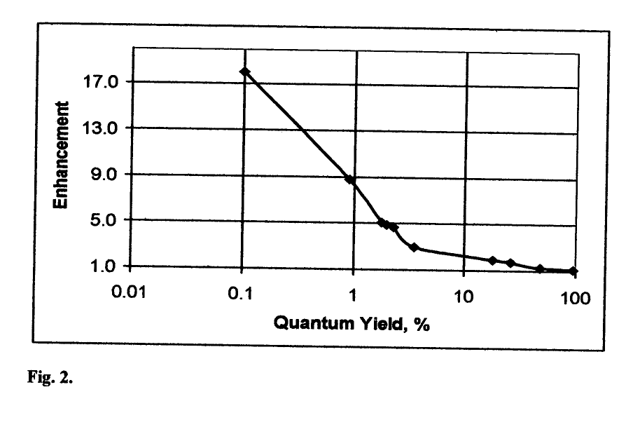

It is proposed by the invention that measurement of multi-band, LES and HES fluorescence enhanced by nearby metal nanoparticles can be used as a novel method to detect an optical signature of sensor and/or analyte. The proposal is based on a discussed above low Q values for non-enhanced HES fluorescence and observed dependence of fluorescence enhance effect on fluorophore quantum yield. In a recent experiment, the emission intensity measured for a series of fluorophores in the vicinity of metal nanoparticles was greatly increasing for the decreasing values of Q for fluorescence (FIG. 2). This result is consistent with findings observed by Lakowicz et al., noting a substantial intrinsic fluorescence enhancement for DNA (Q≈0.01%) at room temperature (J. R. Lakowicz et al, “Intrinsic fluorescence from DNA can be enhanced by metallic particles”, Biochem. Biophys. Res. Comm. 286, 875 (2001)). Without SPR-mediated enhancement, the DNA fluorescence could not be observed at room temperature. Thus, SPR-mediated fluorescence enhancement is a quantum yield dependent effect.

Because quantum yield and lifetime for HES and LES fluorescence of the same fluorophore differ by orders of magnitude, the enhancement effect is expected to be high for a short-living HES (low Q) and low for a long-living LES (high Q) of the same fluorophore. As a result, fluorescence intensities from HES and LES would reach comparable levels. HES fluorescence could then be used as an additional, new measurable optical signature.

In addition to a better specificity, the sensor proposed in this invention has superior conventional sensors in sensitivity. It is a result of enhance fluorophore absorption rate with nearby metal nanoparticles. Absorption rate enhancement is caused by the electro-magnetic (EM) field E generated by surface plasmons in the evanescent zone. A magnitude of SPR EM field exceeds a magnitude EM field of incident light 10² folds. Since the rate of the one-photon excitation is proportional to 1|E|², absorption rate can be enhanced by ˜10⁴ compare to sensors that do not employ SPR.

Enhanced EM field by surface plasmon is especially effective in non-linear, multi-photon excitation. For a two-photon excitation the absorption rate enhancement could be as high as 10⁸ (J. R. Lakowicz, Y. Shen, S. D'Auria, J. Malicka, J. Fang, Z. Gryczynski and I. Gryczynksi, “Radiative Decay Engineering. 2. Effects of Silver Island Films on Fluorescence Intensity, Lifetimes, and Resonance Energy Transfer”, Anal. Biochem., 301:261 (2002)). Metal nanoparticles can also enhance the rate of transient absorption by excited fluorophores in a resonant two-photon HES excitation (M. D. Galanin, and Z. A. Chizhikova, “Fluorescence from the second excited electronic level and absorption by excited R6G molecules”, Bull. Acad. Sc., Phys. Ser. 36, 850 (1972)). The first photon excites the long-living LES and then, the second photon populates the HES through the SPR-enhanced absorption. Such a step-wise two-photon HES excitation (M. D. Galanin, and Z. A. Chizhikova, “Fluorescence from the second excited electronic level and absorption by excited R6G molecules”, Bull. Acad Sc., Phys. Ser. 36, 850 (1972)) is then followed by the SPR-enhanced emission. The resonance- and SPR-enhanced two-photon excitation will greatly increase the intensity of the SPR-enhanced HES emission. This is our concept behind the proposed multi-signature (HES+LES bands) fluorescence sensing.

Two-photon, step-wise HES excitation has been shown to generate a measurable intensity of HES emission and to reduce the background contribution (M. D. Galanin and Z. A. Chizhikova, “Fluorescence from the second excited electronic level and absorption by excited R6G molecules”, Bull. Acad. Sc., Phys. Ser. 36, 850 (1972); Lin, and M. R. Topp, “Low quantum-yield molecular fluorescence: excitation energy dependence and fluorescence polarization in xanthene dyes”, Chem. Phys. Lett. 47, 442 (1977)). An example of HES emission spectrum measured at step-wise excitation of Rhodamine 6G (R6G) solution is shown in FIG. 5. The position of the short-wavelength HES fluorescence band correlates with the position of the absorption band but there is no strict mirror symmetry for these bands. This lack of symmetry is caused by the short HES fluorescence lifetime (0.2 ps for R6G), as the HES decay competes with vibrational relaxation of excited fluorophore (M. D. Galanin and Z. A. Chizhikova, “Fluorescence from the second excited electronic level and absorption by excited R6G molecules”, Bull.

Acad. Sc., Phys. Ser. 36, 850 (1972).

Another surprising feature of HES emission is the dependence of the fluorescence spectrum on energy of the excitation photon, FIG. 6 shows this dependence for HES fluorescence of 1,2-benzanthracene solution (Bogdanov, “Fluorescence and multiwave mixing induced by photon absorption of excited molecules”, Topics in Fluorescence Spectroscopy, Vol. 5: Nonlinear and Two-photon induced Fluorescence, Ed. J. Lakowicz, Plenum Press, 1997). Lowering the excitation energy results in both blue shift and more structure in the HES emission spectrum (the long wavelength LES fluorescence spectrum is independent of the excitation energy, due to a relatively long LES lifetime). These results indicate that the HES fluorescence spectrum should be also more sensitive to the fluorophore's environment and thus provide a more selective spectral signature than the LES spectrum.

This invention also applied to dual sensing of analytes by using surface enhanced multiband fluorescence and surface enhanced multiband Raman scattering.

It will be understood by those skilled in the art that the present invention is a novel and useful method for highly specific, sensitive and fast optochemical sensing.

1. A method and composition of surface plasmon resonance enhanced multiband absorption and multiband fluorescence for optochemical sensing and molecular identification comprises:
a) A molecule, electromagnetic radiation and a metal nanoparticle interacting on each other causing enhanced multiband absorption and multiband emission of the molecule,
b) An analyte chemically or physically interacting with the molecule in the presence of the metal nanoparticle, wherein said the analyte modifies multiband absorption and multiband emission properties of the molecule,
c) A spacer to control distance between the molecule and the metal nanoparticle to optimize multiband absorption and multiband emission from the molecule,
e) A sensor for optochemical sensing of analytes by surface plasmon resonance enhanced multiband absorption and multiband emission of the molecule,
f) An electromagnetic radiation source or chemical source for excitation the molecule and the metal nanoparticle.

2. The method of claim 1, wherein the molecule comprises an organic molecule, inorganic molecule, biomolecule.

3. The method of claim 2, wherein the molecule is fluorophore and is selected from the group consisting of a protein, amino acid, oligonucleotide, lipid, sugar moiety, purine or pyrimidine, nucleoside or nucleotide, genetically engineered biomolecule, fluorescence dye, fluorescence biomarker, metal ligand charge transfer complex, up-converted fluorophore, fluorescence dendrimer, pair of fluorescent donor and fluorescent acceptor, pair of fluorescent donor and quencher, fluorescent metal nanoparticle.

4. The method of claim 1, wherein the analyte is selected from the group consisting of glucose, inorganic molecule, protein, amino acid, oligonucleotide, lipid, sugar moiety, purine or pyrimidine, nucleoside or nucleotide.

5. The method of claim 1, wherein the spacer is selected from the group consisting of a biorecognitive spacer, dielectric spacer, chemical link spacer, analyte sensitive spacer, polymer.

6. The method of claim 1, wherein the metal nanoparticle is a metal, conducting material, super conducting material, semi conducting material.

7. The method of claim 6, wherein the metal is selected from the group consisting of silver, ruthenium, platinum, rhenium, rhodium, osmium, iridium, copper, palladium and gold.

8. The method of claim 1, wherein the metal nanoparticle is sub-wavelength in size.

9. The method of claim 1, wherein the spacer separates the molecule from the metal nanoparticle by distance longer than 10 nm.

10. The method of claims 1, wherein the sensor comprises of the single metal nanoparticles and electromagnetic radiation interacting with molecules at the specific location.

11. The method of claim 1, wherein the sensor comprises at least one thin film of nanoparticles coated on an optical material of refractive index values from 1 to 3.5 and electromagnetic radiation interacting with the molecules and metal nanoparticles.

12. A method of claims 1, for optochemical sensing of the multiband absorption and multiband fluorescence of the molecule, said method comprising the steps of: (a) positioning the nanoparticle and the molecule at a distance apart sufficient to manipulate the multiband fluorescence from the molecule; (b) exposing the molecule to exciting radiation in the single-photon and multi-photons modes of excitation; and (c) analyzing the multiband absorption and multiband fluorescence from the molecule.

13. The method of claims 1, wherein the sensor is a microarray, bio-chip, flow cell, endoscope, microscopic slide, total internal reflection cell, catheter, optical fiber, waveguide.

14. The method of claim 1, wherein the electromagnetic radiation source is selected from the group consisting of a laser with single wavelength, laser with plurality wavelengths, laser diode, light emitted diode, lamp, bioluminescence, chemiluminescence, electroluminescence.

15. The method of claim 1, and 12, wherein the method of optochemical sensing comprises analyses of a low excited state and higher excited states absorption and fluorescence bands of the molecule.

16. The method of claim 1, and 12, wherein the method of molecular identification comprises analyses of the low excited state and higher excited states absorption and fluorescence bands of the molecule.

17. The method of claim 15, and 16, wherein the low excited state and higher excited states absorption and fluorescence bands of the molecule comprises analyses of absorption spectra, fluorescence intensity, fluorescence polarization, fluorescence spectra, hyperspectral imaging, fluorescence lifetime, enhanced Raman scattering, one-photon and multi-photon microscopy, one-photon and multi-photon spectroscopy, fluorescence recovery after photobleaching, fluorescence immunoassay, fluorescence resonance energy transfer.

18. A method of claim 1 for engineering multiband fluorescence lifetime of the molecule by changing the distances of the molecule adjacent to the nanoparticle; and exposing the molecule to an amount of exciting radiation in the single-photon and multi-photons modes of excitation.

19. A method of claim 1 for increasing multiband fluorescence resonance energy transfer on a labeled molecule by changing the distances of the molecule adjacent to a metal particle; and exposing the molecule to an amount of exciting radiation in the single-photon and multi-photons modes of excitation.

20. A method of claim 1 and 12 for optical sensing with multiband emission and multiband absorption of the molecule wherein the analyte sensitive spacer modifies multiband emission and multiband absorption of the molecule.
